(19) United States
(12) Patent Application Publication
(10) Pub. No.: US 2024/0410849 A1
(43) Pub. Date: Dec. 12, 2024

(54) MANUFACTURING MEHTOD AND STRUCTURE OF MICRO HEATING AND SENSING DEVICE

(71) Applicant: Guang-Je Guo, Hsinchu County (TW)

(72) Inventor: Guang-Je Guo, Hsinchu County (TW)

(21) Appl. No.: 18/679,512

(22) Filed: May 31, 2024

(30) Foreign Application Priority Data
Jun. 6, 2023 (TW) 112121139

(51) Int. Cl. G01N 27/12; C23C 14/02; G01K 1/16; G01N 33/00; H05B 3/26

(52) U.S. Cl. CPC G01N 27/12; G01K 1/16; G01N 33/0027; H05B 3/26; C23C 14/021

(57) ABSTRACT

A method of manufacturing a micro heating and sensing device and a device thereof are provided. The micro heating and sensing device includes a substrate, a first electrode layer, a first connection electrode layer, a conductive layer, a second connection electrode layer, and a second electrode layer. By replacement of one end of the metal electrode or the entire metal electrode with conductive oxides or conductive nitrides, problems of micro heating and sensing devices available now including poor contact in electrode connections and needs of passivation layers disposed at the rear end of the electrodes can be overcome. Both cost and time for manufacturing process are reduced.

### FIELD OF THE INVENTION

The present invention relates to a manufacturing method and a structure of a micro heating and sensing device, especially to a manufacturing method and a structure of a micro heating and sensing device using conductive oxides or conductive nitrides to partially or fully replace metal electrode.

### BACKGROUND OF THE INVENTION

Along with commercialization and industrialization of modern society, more and more inner space is built and vehicles are used for people to take breaks, work and commute. However, when people stay in the closed inner space, harmful gases often accumulate in the space due to poor air circulation. The harmful gases at least affect people's quality of life, and worst of all, they become toxic and dangerous to human bodies. Generally, the indoor CO2 levels under 1000 ppm are considered normal with good circulation in indoor environments. When the indoor CO2 levels are increased to 1,000 ppm˜2,000 p, insufficient oxygen makes people feel tired and irritable. When the indoor CO2 levels are further raised to 2,000 ppm˜5,000 ppm, people feel uncomfortable including headaches, sleepiness, poor concentration, loos of attention, increased heart rate and slight nausea. Once the indoor CO2 levels are over 5,000 ppm, exposure may lead to serious oxygen deprivation, resulting in permanent brain damage, coma, even death. While measuring the indoor CO2 levels in our daily lives, the value of the indoor CO2 level measured reaches about 2,000 ppm˜3,000 ppm due to poor indoors air circulation or too many people in certain space. People start to get sleepy and feel a bit discomfort. If no measure is taken to control the indoor CO2 levels, the indoor CO2 levels may keep increasing and people in the space are exposed to hazards.

Owing to the rise of Internet of Things (IoT) and progress in environmental sensing technology, demand for gas sensing technology is growing. The gas sensing technology can be applied to monitoring different environments including home environment, offices, factories, medical institutions, etc.

The conventional gas sensors have wide applications, not matter in industry or at home. The resistance-type gas sensor uses metal electrodes to heat heating plates made of metal oxides. When oxygen in air is in contact with the metal oxides (such as tin(IV) oxide), the oxygen takes electrons away from the metal oxides so that electrical resistance raises. When gas in air users intend to detect (such as flammable gas) are getting closer, it reacts with oxygen around the sensor and the electrons are returned to the metal oxides so that electrical resistance decreases. Now users learn that there is the gas users intend to detect. In such sensing way, the gas users intend to detect directly reacts with the oxygen surrounding the sensor instead of the metal oxides. Thereby stability of the gas sensor after long term use is improved. Later the development of the gas sensor is nothing more than better heating materials or additives while there is no significant change in basic principles.

In recent years, the environmental sensors integrated with multiple functions are developed. For compact design, most of gas sensors are produced using MEMS (Micro Electro Mechanical Systems) technology.

The MEMS is a process technology developed by combination of microelectronics with mechanical engineering and used to create tiny devices with sizes ranging from micrometer (μm) to millimeter (mm). Generally, MEMS is a tiny device formed by integration of mechanical parts, electronic circuit, sensors and actuators onto a substrate and produced using similar processes as those used in the fabrication of semiconductor.

Wherein, the micro heating and sensing device is a tiny temperature control unit including at least two metal electrodes to heat a resistance material at a center thereof. Thus, resistivity changes due to the temperature changes, or the resistance material reacts with substances outside. The micro heating and sensing device are not only applied to gas sensors and temperature sensors but also temperature sensing and control units in components and microreactors.

In conventional micro heating and sensing devices, extremely thin metal electrodes are used in order to prevent heat conduction during fabrication of the electrodes. At the same time, a surface of the electrode is covered with a passivation layer made of insulation materials to reduce poisoning of the electrode caused by contact with external gases for protection of the electrode.

In order to coating the passivation layer on the surface of the electrode, surface treatment is carried out after a plurality of times of deposition of the insulation materials and metal sputtering or evaporation during manufacturing process. Yet the complicated manufacturing process leads to significantly increased cost and production time.

The arrangement of the passivation layer(s) is easy to make a heating layer used for sensing/temperature control and the extremely-thin metal electrode have level difference between them and further cause poor contact.

Thus, there is room for improvement and there is a need to provide a novel improved manufacturing process of the surface of the heating electrode with temperature control capability.

### SUMMARY

Therefore, a primary object of the present invention is to provide a manufacturing method of a micro heating and sensing device, in which additional conductive substances are adopted as a circuit connection between two metal heating electrodes and both ends of a conductive layer for sensing or temperature-control. Thereby, the structure of the heating electrodes is improved and the heating electrodes have both high electrical conductivity and low thermal conductivity. At the same time, abilities of measurement, temperature control and thermal insulation for the heating electrode are also maintained.

A secondary object of the present invention is to provide a structure for a micro heating and sensing device in which heating electrodes are produced by conductive oxides or conductive nitrides. By replacement of the whole metal electrodes with the electrodes made of conductive oxides or conductive nitrides, the manufacturing process is simplified.

Another primary object of the present invention is to provide a micro heating and sensing device in which additional conductive substances are used for circuit connection between two metal heating electrodes and one end of a sensing or temperature-control conductive layer in contact with the electrodes. Thereby structure of the heating electrodes is improved and the heating electrodes have both high electrical conductivity and low thermal conductivity. At the same time, measurement ability, temperature control ability and thermal insulation ability of the heating electrode are also maintained.

It is another secondary object of the present invention to provide a micro heating and sensing device in which insulation materials covering an outer surface of metal electrodes and conductive layers are used as passivation layers to reduce poisoning of the electrode caused by effects of gas on the metal electrodes and the conductive layer while performing gas sensing.

In order to achieve the above primary objects, a method of manufacturing a micro heating and sensing device according to the present invention includes the following steps. First disposing a sacrificial layer on a substrate. Then etching the sacrificial layer to form a first hole and a second hole spaced apart from each other and both penetrating the sacrificial layer to an upper surface of the substrate. Disposing a first passivation layer on the sacrificial layer, removing the first passivation layer on a bottom of the first hole and a bottom of the second hole by etching, and disposing a first electrode layer and a second electrode layer respectively on the first hole and the second hole. Next covering the first electrode layer and the second electrode layer with a second passivation layer. Then mounting a conductive layer over an interval space between the first electrode layer and the second electrode layer and the conductive layer located on the first passivation layer. Later covering the conductive layer with a third passivation layer. Removing a part of the third passivation layer on the conductive layer by etching to form a first port and a second port. Also removing a part of the second passivation layer on the first electrode layer and the second electrode layer respectively by etching to form a third port and a fourth port. Then disposing a first connection electrode layer and a second connection electrode layer on both the second passivation layer and the third passivation layer and the first connection electrode layer and the second connection electrode layer are spaced apart from each other. The first connection electrode layer is coupled to the conductive layer and the first electrode layer respectively through the first port and the third port. The second connection electrode layer is coupled to the conductive layer and the second electrode layer respectively through the second port and the fourth port. Lastly, removing the sacrificial layer, the first passivation layer located outside the first hole and covering the sacrificial layer, and the first passivation layer located outside the second hole and covering the sacrificial layer by etching.

Preferably, after the step of removing the first passivation layer on a bottom of the first hole and a bottom of the second hole by etching and disposing a first electrode layer and a second electrode layer respectively on the first hole and the second hole, the method further includes a step of etching the first electrode layer and the second electrode layer respectively to form a first recess and a second recess.

Preferably, the first connection electrode layer and the second connection electrode layer are made of a material selected from the group consisting of indium tin oxide (ITO), zinc oxide (ZnO), indium zinc oxide (IZO), aluminum gallium oxide (AGO), aluminum-doped zinc oxide (AZO), titanium nitride (TiN), tantalum nitride (TaN), aluminum (Al), titanium (Ti), and a combination thereof.

In order to achieve the above secondary objects of the present invention, a material for both the first electrode layer and the second electrode layer is selected from the group consisting of gold (Ag), silver (Au), platinum (Pt), aluminum (Al), titanium (Ti), tantalum (Ta), indium tin oxide (ITO), zinc oxide (ZnO), indium zinc oxide (IZO), aluminum gallium oxide (AGO), aluminum-doped zinc oxide (AZO), titanium nitride (TiN), tantalum nitride (TaN), and a combination thereof.

In order to achieve another primary object of the present invention, a micro heating and sensing device according to the present invention includes a substrate, a first electrode layer, a first connection electrode layer, a conductive layer, a second connection electrode layer, and a second electrode layer. The first electrode layer is disposed on the substrate and the first connection electrode layer is disposed on and coupled to the first electrode layer. The first connection electrode layer is extending horizontally. The conductive layer is disposed under and coupled to the first connection electrode layer. The conductive layer and the first electrode layer are spaced apart from each other and an air layer is formed between the conductive layer and the substrate. The second connection electrode layer is disposed on and coupled to the conductive layer. The first connection electrode layer and the second connection electrode layer are spaced apart from each other while the second electrode layer is mounted on the substrate and coupled under the second connection electrode layer. During electrical conduction process, a current is flowing from the first electrode layer to the first connection electrode layer, then through the conductive layer, the second connection electrode layer, and the second electrode layer in sequence, and finally flowing to the substrate.

Preferably, the first connection electrode layer and the second connection electrode layer are made of a material selected from the group consisting of indium tin oxide (ITO), zinc oxide (ZnO), indium zinc oxide (IZO), aluminum gallium oxide (AGO), aluminum-doped zinc oxide (AZO), titanium nitride (TiN), tantalum nitride (TaN), aluminum (Al), titanium (Ti), and a combination thereof.

Preferably, a material for both the first electrode layer and the second electrode layer is selected from the group consisting of gold (Ag), silver (Au), platinum (Pt), aluminum (Al), titanium (Ti), tantalum (Ta), indium tin oxide (ITO), zinc oxide (ZnO), indium zinc oxide (IZO), aluminum gallium oxide (AGO), aluminum-doped zinc oxide (AZO), titanium nitride (TiN), tantalum nitride (TaN), and a combination thereof.

In order to achieve the another secondary object of the present invention, the micro heating and sensing device further includes at least one passivation layer which is an insulation material covering the first electrode layer, the second electrode layer, and the conductive layer. The first passivation layer is made of materials selected from the group consisting of aluminum oxide (Al₂O₃), silicon nitride (Si₃N₄), silicon dioxide (SiO₂), titanium dioxide (TiO₂), and a combination thereof.

Preferably, a lower part of the first electrode layer is extending horizontally toward a first side and then extending upward while a lower part of the second electrode layer is extending horizontally toward a second side and then extending upward.

### BRIEF DESCRIPTION OF THE DRAWINGS

FIG. 1 is a flow chart showing steps of a method of manufacturing micro heating and sensing devices of an embodiment according to the present invention;

FIG. 2A to FIG. 2K are schematic drawings showing sectional views of a part of steps of an embodiment according to the present invention;

FIG. 2L is a schematic drawing showing a current flow of an embodiment according to the present invention;

FIG. 2M is a top view of an embodiment according to the present invention;

FIG. 3A is a step of etching electrodes of another embodiment according to the present invention;

FIG. 3B is a schematic drawing showing a sectional view of another embodiment according to the present invention;

FIG. 3C is a sectional view of another embodiment according to the present invention;

FIG. 3D is a schematic drawing showing a current flow of another embodiment according to the present invention;

FIG. 4A is a sectional view of a further embodiment according to the present invention;

FIG. 4B is a schematic drawing showing a current flow of a further embodiment according to the present invention;

FIG. 4C is a top view of a further embodiment according to the present invention;

FIG. 5 is a top view of a fourth embodiment according to the present invention;

FIG. 6A is a sectional view of a fifth embodiment according to the present invention;

FIG. 6B is a sectional view of a fifth embodiment with passivation layers according to the present invention.

### DETAILED DESCRIPTION

In order to learn features and functions of the present invention more clearly and completely, please refer to the following embodiments and related detailed descriptions.

In the method and structure of a micro heaters and sensor available now, a passivation layer covering its surface is used to prevent electrode poisoning caused by direct contact between metal electrodes and gases outside. However, cost is increased and production time is extended due to multiple times of deposition for coating of the metal electrodes during semiconductor process.

In a micro heating and sensing device of the present invention, additional conductive substances are used for circuit connection between two metal heating electrodes and one end of a sensing or temperature-control conductive layer in contact with the electrodes. Thereby structure of the heating electrodes is improved and the heating electrodes have both high electrical conductivity and low thermal conductivity. At the same time, measurement ability, temperature control ability and thermal insulation ability of the heating electrode are also maintained.

In the following descriptions, a plurality of embodiments is used to describe the present invention in detail, not intended to limit the present invention.

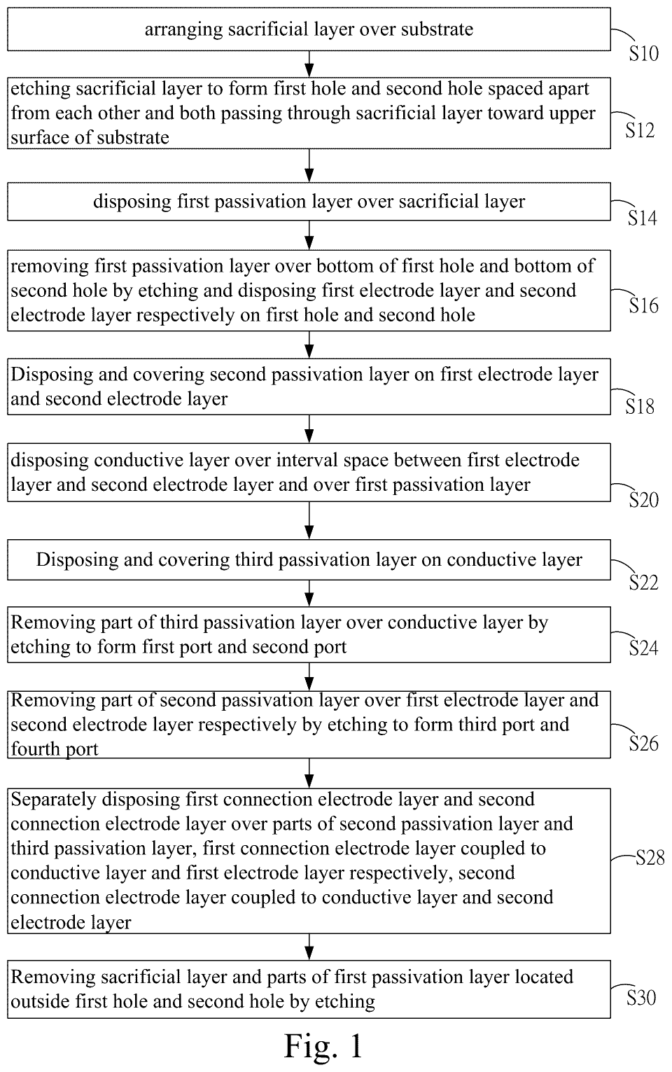

Refer to FIG. 1, a flow chart showing steps of a method of manufacturing a micro heating and sensing device according to the present invention is provided. As shown in FIG. 1, the method includes the following steps.
Step S10: disposing sacrificial layer on substrate;
Step S12: etching sacrificial layer to form first hole and second hole spaced apart from each other and both passing through sacrificial layer toward upper surface of substrate;
Step S14: disposing first passivation layer on sacrificial layer;
Step S16: removing first passivation layer on bottom of first hole and bottom of second hole by etching and disposing first electrode layer and second electrode layer respectively on first hole and second hole;
Step S18: disposing and covering second passivation layer on first electrode layer and second electrode layer;
Step S20: disposing conductive layer on interval space between first electrode layer and second electrode layer and on first passivation layer;
Step S22: disposing and covering third passivation layer on conductive layer;
Step S24: removing part of third passivation layer on conductive layer by etching to form first port and second port;
Step S26: removing part of second passivation layer on first electrode layer and second electrode layer respectively by etching to form third port and fourth port;
Step S28: separately disposing first connection electrode layer and second connection electrode layer on part of second passivation layer and part of third passivation layer, the first connection electrode layer coupled to the conductive layer and first electrode layer respectively, second connection electrode layer coupled to conductive layer and second electrode layer respectively; and
Step S30: removing sacrificial layer and part of first passivation layer located outside first hole and second hole by etching.

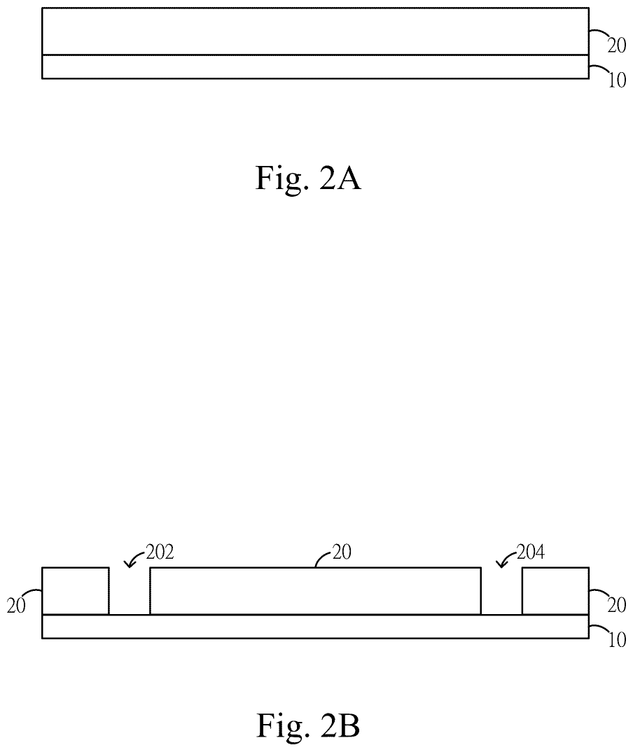

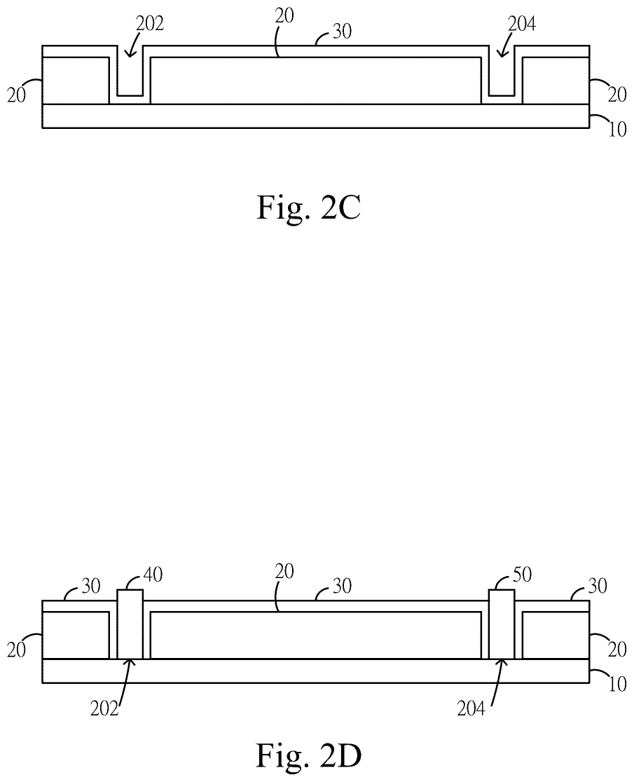

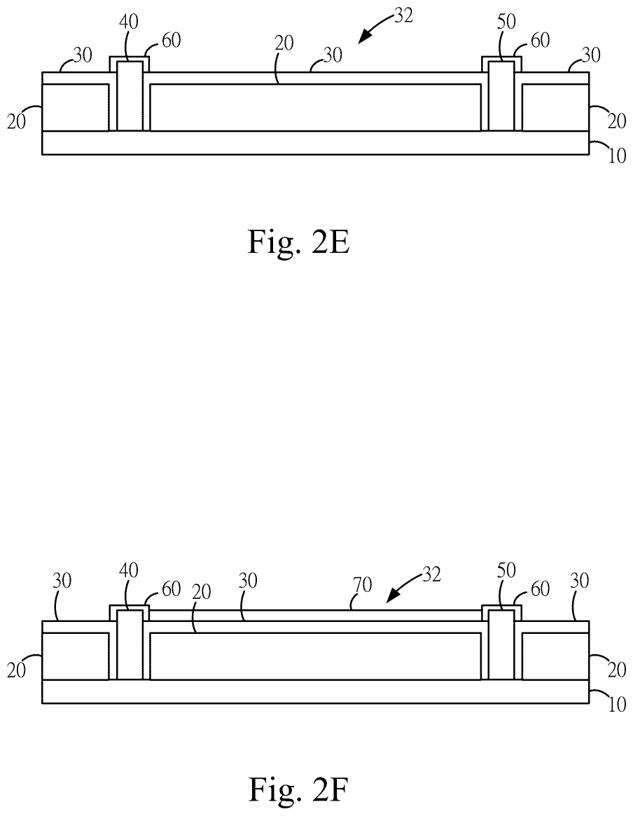

Refer to FIG. 2A to FIG. 2K, schematic drawings showing sectional views of a part of steps of an embodiment according to the present invention are provided.

As shown in FIG. 2A, in the step S10, a substrate 10 is selected and a sacrificial layer 20 is deposited or coated on an upper surface of the substrate 10. Since the micro heating and sensing device needs to have current flowing through it to generate heat, a printed circuit is disposed on a surface of the substrate 10 by printing before deposition or coating of the sacrificial layer 20.

A material for the substrate 10 is selected from the group consisting of silicon (Si), silicon dioxide (SiO₂), silicon nitride (Si₃N₄), and a combination thereof. The above materials are all silicon-based materials with low thermal conductivity.

The sacrificial layer 20 is made of phosphosilicate glass (PSG), borophosphosilicate glass (BPSG), photoresists, or high molecular weight polymer. An ideal material for the sacrificial layer 20 must meet the following requirements. First a thickness of the sacrificial layer 20 must be controlled within an acceptable tolerance because that non-uniform deposition leads to a rough or uneven surface of mechanical parts. While releasing parts of a structural layer, the sacrificial layer 20 should be removed clearly. Moreover, the sacrificial layer 20 must have high etch selectivity and etch rate so that other parts of the structure will not get damaged significantly during etching of the sacrificial layer 20. PSG or BPSG with loose structure can be removed clearly in hydrofluoric acid (HF) at a faster etching rate, without causing other damages to the structure. In a preferred embodiment, the photoresists and high molecular weight polymers can be removed using oxygen plasma etching which will not cause other damages to the structure due to its low etching efficiency for conductive metal oxides and metal nitrides.

Refer to FIG. 2B, in the step S12, an upper surface of the sacrificial layer is etched by dry etching (plasma etch) or other anisotropic etching to form a first hole 202 and a second hole 204 which are spaced apart from each other and penetrating the sacrificial layer 20 to an upper surface of the substrate 10. The first hole 202 and the second hole 204 are used for mounting the following structure.

As shown in FIG. 2C, in the step S14, a first passivation layer 30 is deposited on the sacrificial layer 20. The deposition of the first passivation layer 30 has two main purposes. First purpose is to protect electrodes from corrosion caused by external gases during heating process. The second is to prevent the electrodes from electrical leakage by using electrical insulation materials as passivation materials for the first passivation layer 30.

The first passivation layer 30 is made of a material selected from the group consisting of aluminum oxide (Al₂O₃), silicon nitride (Si₃N₄), silicon dioxide (SiO₂), and titanium dioxide (TiO₂), and a combination thereof, which are all electrical insulation materials.

In the step S16, as shown in FIG. 2D, parts of the first passivation layer 30 in part of the first hole 202 and in part of the second hole 204 by etching (such as dry etching (plasma ion bombardment plasma etch) or other anisotropic etching). A first electrode layer 40 and a second electrode layer 50 are respectively disposed in the first hole 202 and the second hole 204 to be extended outward excessing the first passivation layer 30 on the sacrificial layer 20. Thereby, a height of both the first electrode layer 40 and the second electrode layer 50 is higher than a top surface of the first passivation layer 30. The first electrode layer 40 and the second electrode layer 50 are usually disposed at a position required by sputtering or evaporation of precious metals. Then, the rest parts are removed by acid, alkali, or plasma dry etching. Although the precious metals are unable to be deposited by semiconductor processes, gas sensors have better performance while detecting their resistance due to their excellent electrical properties. In this step, conductive oxides or conductive nitrides are deposited on the first hole 202 and the second hole 204 to form a first electrode layer 40 and a second electrode layer 50 respectively. A redundant part is removed by etching. The formation of the electrode layer by deposition can reduce manufacturing tolerance and time. During sensing of gases, the conductive oxides or nitrides are not easily affected by the gases and no significant electrode poisoning occurs. Electrode poisoning is the reduction of efficiency on the electrode surface due to deposition of reactants. A surface of the electrode is contaminated or covered with oxide or nitrides during electrochemical process so that electrode activity is lowered and the electrode efficiency is reduced. Thereby the electrochemical reaction is affected. Moreover, when metals including aluminum, titanium, tantalum, etc. is exposed in high temperature air, a layer of oxidized protective film is formed on surface of the metal to prevent metal inside from interaction with the surroundings.

The first electrode layer 40 and the second electrode layer 50 both are made of a material selected from the group consisting of gold (Ag), silver (Au), platinum (Pt), aluminum (Al), titanium (Ti), tantalum (Ta), indium tin oxide (ITO), zinc oxide (ZnO), indium zinc oxide (IZO), aluminum gallium oxide (AGO), aluminum-doped zinc oxide (AZO), titanium nitride (TiN), tantalum nitride (TaN), and a combination thereof.

In the step S18, as shown in FIG. 2E, the first electrode layer 40 and the second electrode layer 50 are respectively covered with a second passivation layer 60 which is made of material the same as that for the first passivation layer 30. That is, two of the second passivation layer 60 are separately disposed on the first electrode layer 40 and the second electrode layer 50. The first passivation layer 30 and the second passivation layer 60 are covering the first electrode layer 40 and the second electrode layer 50. An area between the first electrode layer 40 and the second electrode layer 50 is removed by etching for following arrangement of other parts conveniently.

As shown in FIG. 2F, in the step S20, a conductive layer 70 is deposited on an interval space 32 between the first electrode layer 40 and the second electrode layer 50 with the second passivation layer 60 between the first electrode layer 40 and the conductive layer 70 as well as the second passivation layer 60 between the second electrode layer 50 and the conductive layer 70. The conductive layer 70 is located on the first passivation layer 30 and a redundant part is removed. In the micro heating and sensing device, circuit is disposed on a surface of the conductive layer 70 by printing and a current is flowing through the circuit to perform heating according to materials for the conductive layer 70. Resistance of the materials for the conductive layer 70 varies according to temperature or chemical reactions. The changes in resistance of the conductive layer 70 is measured by the following circuit through the electrodes so as to carry out sensing. A temperature control method is to change temperature according to current amplitude input into the conductive layer 70.

A material for the conductive layer 70 is selected from the group consisting of gold (Au), aluminum (Al), titanium (Ti), vanadium (V), nickel (Ni), Tungsten (W), titanium nitride (TiN), tantalum nitride (TaN), polyacetylene (PA), graphene, polycrystalline silicon, and a combination thereof.

Refer to FIG. 2G, in the step S22, a third passivation layer 80 made of the same material as the first passivation layer 30 is deposited on and covering a surface of the conductive layer 70 and the third passivation layer 80 is at the same level as the second passivation layer 60. The first passivation layer 30, the second passivation layer 60, and the third passivation layer 80 cover the conductive layer 70.

Refer to FIG. 2H, in the step S24, a part of the third passivation layer 80 on the conductive layer 70 is removed by dry etching (plasma ion bombardment) or other anisotropic etching to form a first port 802 and a second port 804.

As shown in FIG. 21, in the step S26, a part of the second passivation layer 60 on the first electrode layer 40 and a part of the second passivation layer 60 on the second electrode layer 50 are respectively removed by etching (such as, dry etching (plasma etch) or other anisotropic etching) to form a third port 806 and a fourth port 808.

In the step S28, as shown in FIG. 2J, a connection electrode layer is deposited on the second passivation layer 60 and the third passivation layer 80 and then etched to form a first connection electrode layer 92 and a second connection electrode layer 94. The first connection electrode layer 92 is coupled to the conductive layer 70 and the first electrode layer 40 respectively through the first port 802 and the third port 806. The second connection electrode layer 94 is coupled to the conductive layer 70 and the second electrode layer 50 respectively through the second port 804 and the fourth port 808. A circuit is formed on a surface of the first connection electrode layer 92 for conducting the first electrode layer 40 and the conductive layer 70. A circuit is also formed on a surface of the second connection electrode layer 94 for conducting the second electrode layer 50 and the conductive layer 70.

Both the first connection electrode layer 92 and the second connection electrode layer 94 are made of a material selected from the groups consisting of indium tin oxide (ITO), zinc oxide (ZnO), indium zinc oxide (IZO), aluminum gallium oxide (AGO), aluminum-doped zinc oxide (AZO), titanium nitride (TiN), tantalum nitride (TaN), aluminum (Al), titanium (Ti), and a combination thereof.

In the above embodiment, one end of the metal electrode is replaced by conductive oxides or conductive nitrides. Thus damages or modifications of metals of the metal electrode caused by use of semiconductor processes including deposition, wet etching, plasma ion bombardment, etc. can be solved. Both difficulties of manufacturing processes and production time are reduced. There is no need to dispose a passivation layer at a surface of the conductive oxides or conductive nitrides. The structural design of the heating electrode is further simplified to reduce the difficulties of the manufacturing processes.

As shown in FIG. 2K, in the step S30, removing the sacrificial layer 20, the first passivation layer 30 located outside the first hole 202 and covering the sacrificial layer 20, and the first passivation layer 30 located outside the second hole 204 and covering the sacrificial layer 20 by dry etching (plasma ion bombardment) or other anisotropic etching; removing the sacrificial layer 20 under the conductive layer 70 to form an air layer 22 which provides good thermal insulation effect and increase contact area between the conductive layer 70 and outside air; thereby sensor accuracy is improved while the present device being applied to gas sensors and temperature sensors.

Refer to FIG. 2L and FIG. 2M, a current flow and a top view of an embodiment according to the present invention are provided. After the above steps S10-S30, as shown in figures, there is no passivation layer drawn in FIG. 2M in order to show a conductive path. During working process of a micro heating and sensing device 1, a current I is flowing from a first printed circuit 12 on the substrate 10, through the first electrode layer 40, the first connection electrode layer 92, a second printed circuit 72 of the conductive layer 70, the second connection electrode layer 94, and the second electrode layer 50 to the first printed circuit 12 on the substrate 10. The micro heating and sensing device 1 can receive signals generated due to resistance changes caused by sensing of gases and temperature, or control temperature required therein according to amplitude of the current.

In short, in a first embodiment of a method of manufacturing a micro heating and sensing device according to the present invention, one end of the metal electrode or the whole metal electrode is replaced by conductive oxides or conductive nitrides instead of a heating electrode made of signal precious metals in conventional manufacturing process. Thereby difficulties of manufacturing processes of the electrode are minimized and production time is reduced. There is no need to dispose the passivation layer on the surface of the conductive oxides or nitrides so that sputtering process required is reduced and the difficulties of the manufacturing processes are further reduced.

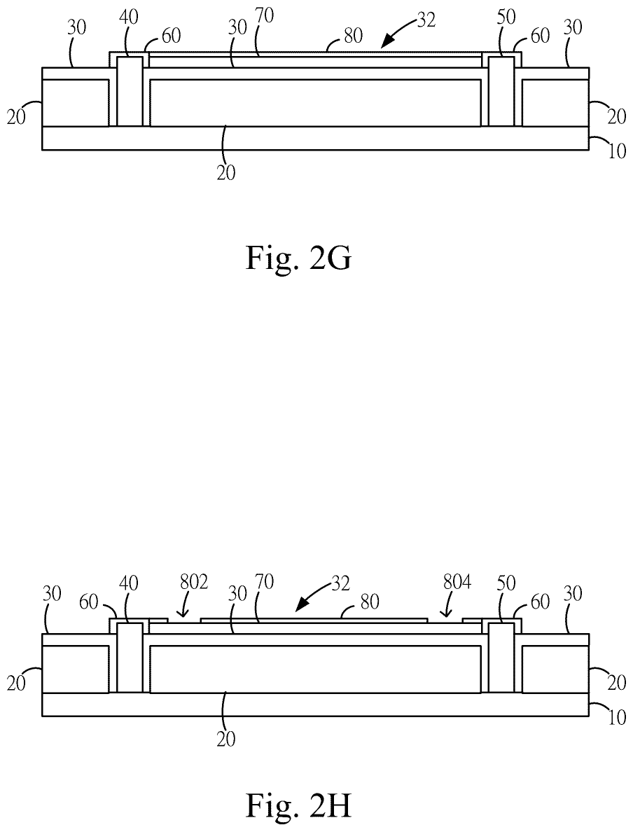

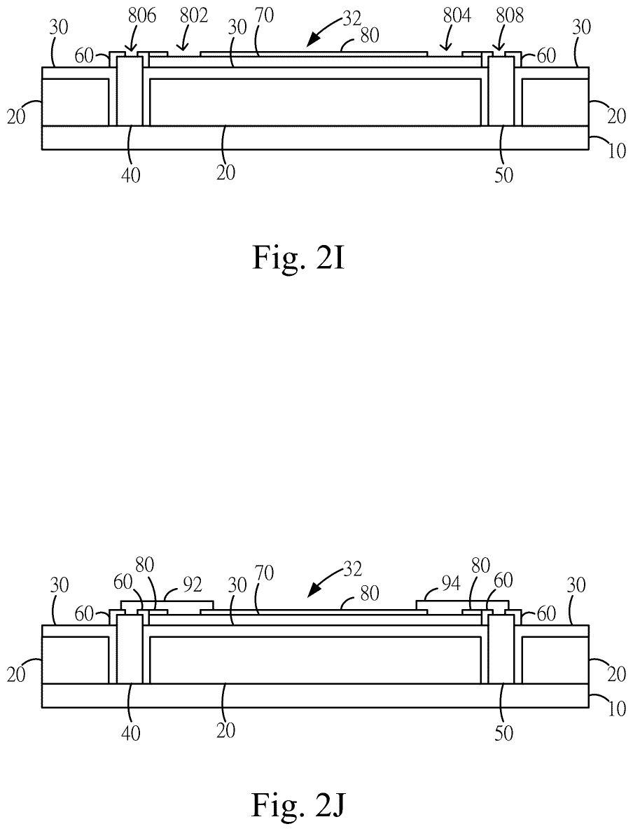

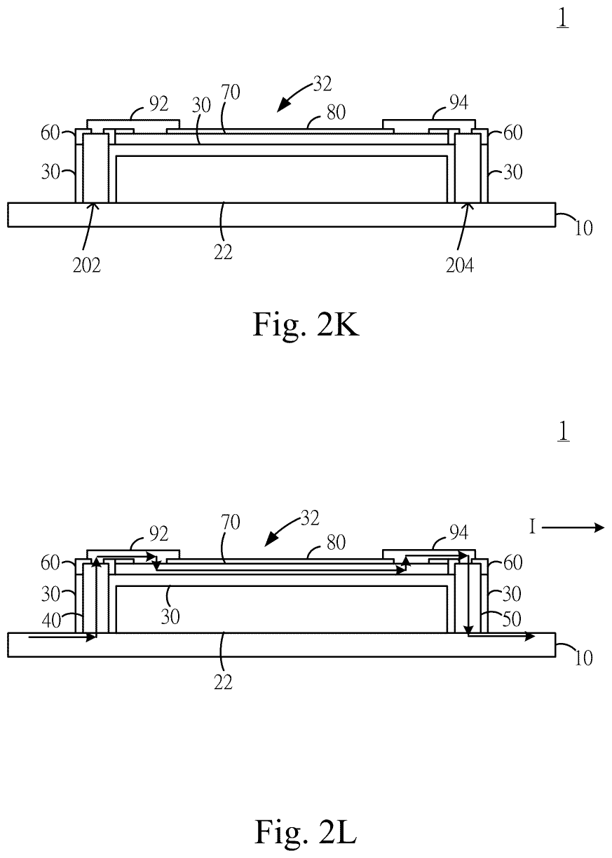

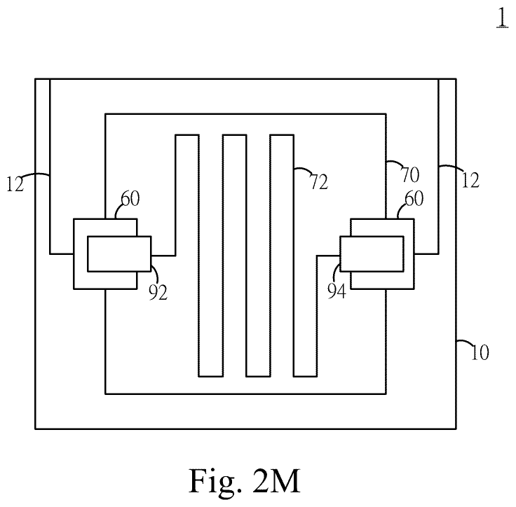

The metal electrode itself is a good conductor. In order to reduce effects of heat loss caused by the metal electrode on the micro heating and sensing device, a further embodiment which is formed based on the first embodiment and shown in FIG. 3A is provided. In this embodiment, the method further includes the following step after the step S16 of the first embodiment.

Step S162: etching the first electrode layer and the second electrode layer respectively to form a first recess and a second recess.

Also refer to FIG. 3B, a schematic drawing showing a sectional view of etched electrodes. As shown in FIG. 3B, in the step S162, an upper surface of first electrode layer 40 and an upper surface of the second electrode layer 50 are respectively etched by physical etching or chemical etching using acids or bases to form a first recess 402 and a second recess 502 correspondingly.

Refer to FIG. 3C, a sectional view of another embodiment is provided. In this embodiment, besides one end of the metal electrode or the whole metal electrode is replaced by conductive oxides or conductive nitrides, the first electrode layer 40 and the second electrode layer 50 are respectively treated by etching to have the first recess 402 and the second recess 502 on their electrode layers, which work like the air layer 22 mentioned above to further reduce temperature loss due to heat conducted by the metal electrodes.

Refer to FIG. 3D, a schematic drawing showing flowing of a current is provided. During working process of a micro heating and sensing device 1, a current I is flowing from a substrate 10, through the first electrode layer 40, the first connection electrode layer 92, the conductive layer 70, the second connection electrode layer 94, and the second electrode layer 50, to the substrate 10. The micro heating and sensing device 1 can receive signals generated due to resistance changes caused by sensing of gases and temperature, or control temperature required therein according to amplitude of the current I.

In a second embodiment of a method of manufacturing a micro heating and sensing device, recesses are further disposed on the electrodes to reduce heat loss caused by the metal electrode on the micro heating and sensing device 1 and further increase temperature control capability of the micro heating and sensing device 1, besides the replacement of one end of the electrode or the whole electrode with conductive oxides or nitrides to reduce difficulties of manufacturing processes of the electrode and production time in the first embodiment.

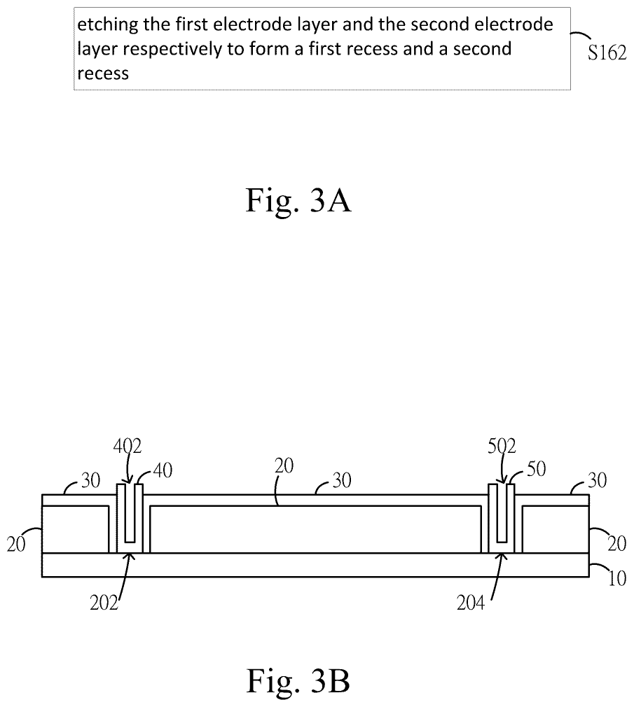

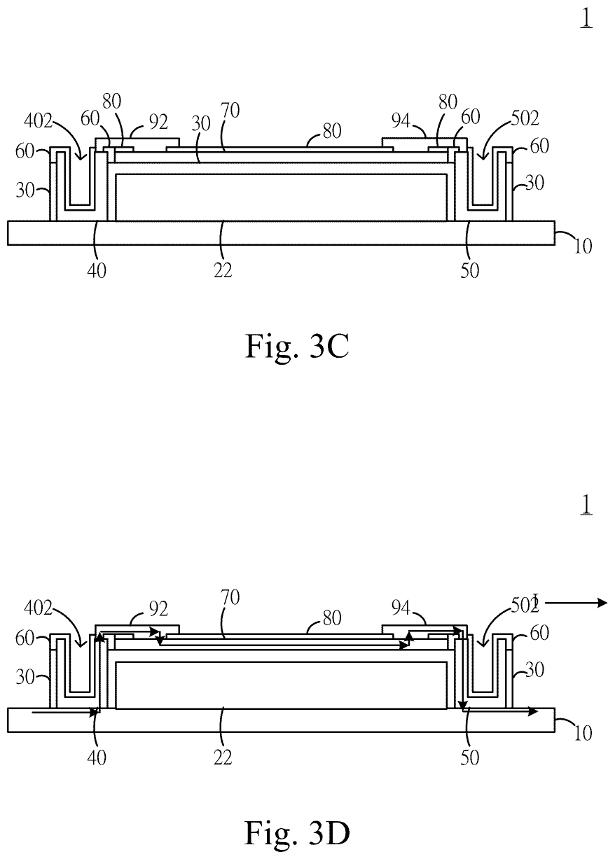

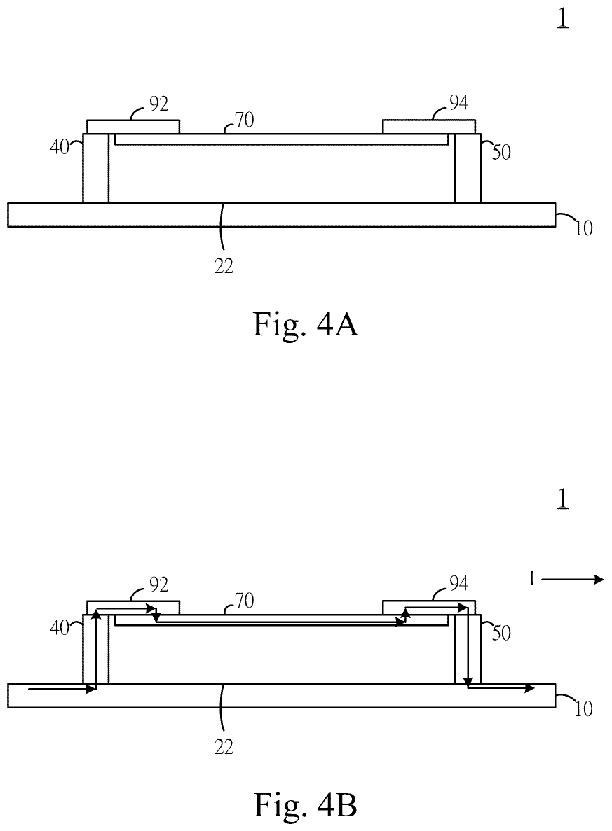

Refer to FIG. 4A, a sectional view of a third embodiment of a micro heating and sensing device according to the present invention is provided. The third embodiment of the present invention provides structure of a micro heating and sensing device. In this embodiment, a micro heating and sensing device 1 includes a substrate 10, a first electrode layer 40, a first connection electrode layer 92, a conductive layer 70, a second connection electrode layer 94, and a second electrode layer 50. The first electrode layer 40 is disposed on an upper surface of the substrate 10 and the first connection electrode layer 92 is disposed at and coupled to an upper surface of the first electrode layer 40. The first connection electrode layer 92 is extending toward one side horizontally. The conductive layer 70 is disposed on and coupled to a lower surface of the first connection electrode layer 92 and extending toward the side with the first electrode layer 40 horizontally. The conductive layer 70 and the first electrode layer 40 are spaced apart from each other. An air layer 22 is formed between the conductive layer 70 and the substrate 10. The second connection electrode layer 94 is disposed at and coupled to an upper surface of the conductive layer 70 and is also extending toward the side with the second electrode layer 50 horizontally. The first connection electrode layer 92 and the second connection electrode layer 94 are spaced apart from each other while the second electrode layer 50 is not only mounted to an upper surface of the substrate 10, but also disposed on and coupled to a lower surface of the second connection electrode layer 94. During the electrical conduction process, a current I is flowing from the first electrode layer 40 to the first connection electrode layer 92, then through the conductive layer 70, the second connection electrode layer 94 and the second electrode layer 50 in sequence, and finally flowing to the substrate 10.

Still refer to FIG. 4A, in the third embodiment, the substrate 10 is made of at least one material selected from the group consisting of silicon (Si), silicon dioxide (SiO₂), silicon nitride (Si₃N₄), and a combination thereof, which are all silicon-based materials with low thermal conductivity. These materials are convenient to obtain during microelectromechanical systems (MEMS) process. Owing to low thermal conductivity, these materials are especially suitable for use in temperature control units such as the micro heating and sensing device.

In the third embodiment, input and output positions of the current I of the micro heating and sensing device are positioned on a surface of the substrate 10 by the printed circuit technique. Then deposit or coat a sacrificial layer and form holes on the sacrificial layer to correspond to the input and output positions. Next form the first electrode layer 40 and the second electrode layer 50 both made of metal by sputtering or evaporation. Or form the first electrode layer 40 and the second electrode layer 50 both made of conductive oxides or conductive nitrides by deposition. The metal electrodes provide better electrical and thermal conductivities. Although the metal electrodes are unable to be deposited by semiconductor processes, gas sensors have better performance during detection of their resistance due to their excellent electrical properties. The electrodes made of conductive oxides or conductive nitrides is easier to be produced. The manufacturing tolerance and time are both reduced when the electrode layer is produced by deposition. During sensing of gases, the conductive oxides or nitrides are not easily affected by the gases and no significant electrode poisoning occurs. Electrode poisoning is the reduction of efficiency on the electrode surface due to deposition of reactants. A surface of the electrode is contaminated or covered with oxide or nitrides during electrochemical process so that electrode activity is lowered and the electrode efficiency is reduced. Thereby the electrochemical reaction is affected.

Next the conductive layer 70 is formed between and spaced apart from the first electrode layer 40 and the second electrode layer 50 by deposition. The conductive layer 70 is disposed on the sacrificial layer between the two electrodes and a conductive path of the current I is mounted on the conductive layer 70 by the printed circuit technique. Then the first connection electrode layer 92 and the second connection electrode layer 94 are disposed thereover. Use conductive oxides or conductive nitrides as a rear end of the metal electrode can reduce reaction probability of metal with gases. That means probability of occurrence of the electrode poisoning mentioned above is reduced. The rear end of the electrode made of the conductive oxides or conductive nitrides can reduce extra heat caused by defects generated by connection of two different materials during the electrical conduction process. The extra heat affects accuracy of the temperature control. Lastly, remove the sacrificial layer to complete production of the micro heating and sensing device 1. The conductive layer 70 is suspended in the air to form an air layer 22 which provides good thermal insulation effect and increase contact area between the conductive layer 70 and air outside to improve sensor accuracy while being applied to gas sensors.

Refer to FIG. 4B and FIG. 4C, a schematic drawing showing flowing of a current in a third embodiment and a top view showing structure of a third embodiment are provided. During the electrical conduction process, starting from the printed circuit of the substrate 10, the current I is then flowing from the first electrode layer 40, through the first connection electrode layer 92, the conductive layer 70, the second connection electrode layer 94, and the second electrode layer 50 in turn, and finally back to the printed circuit of the substrate 10. While being applied to gas sensors or temperature sensors, the micro heating and sensing device 1 can receive the current I from the sensor and sends signals generated due to resistance changes of the conductive layer 70 back to the gas sensor or temperature sensor during gas sensing. When the micro heating and sensing device 1 is mounted in a temperature control device, temperature required in the temperature control device can be controlled by amplitude of the current.

A material for both the first electrode layer 40 and the second electrode layer 50 is selected from the group consisting of gold (Ag), silver (Au), platinum (Pt), aluminum (Al), titanium (Ti), tantalum (Ta), indium tin oxide (ITO), zinc oxide (ZnO), indium zinc oxide (IZO), aluminum gallium oxide (AGO), aluminum-doped zinc oxide (AZO), titanium nitride (TiN), tantalum nitride (TaN), and a combination thereof.

Both the first connection electrode layer 92 and the second connection electrode layer 94 are made of a material selected from the groups consisting of indium tin oxide (ITO), zinc oxide (ZnO), indium zinc oxide (IZO), aluminum gallium oxide (AGO), aluminum-doped zinc oxide (AZO), titanium nitride (TiN), tantalum nitride (TaN), aluminum (Al), titanium (Ti), and a combination thereof.

Therefore, the third embodiment of the present invention provides a micro heating and sensing device in which one end of the electrode or the whole electrode is made of conductive oxides or conductive nitrides instead of metal to reduce difficulties of manufacturing processes, production time required, and sputtering processes needed.

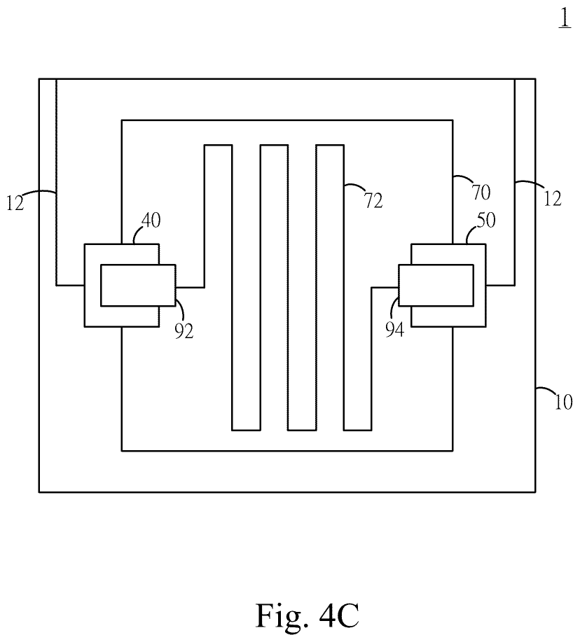

In order to prevent the electrode layer from being affected by external air and impurities, refer to FIG. 5, a sectional view showing structure of a fourth embodiment is provided. Based on the third embodiment mentioned above, the fourth embodiment of the micro heating and sensing device 1 further includes a passivation layer 110 which is disposed on and covering the first electrode layer 40, the second electrode layer 50, and the micro heating and sensing device 1 and made of electrical insulation materials.

Still refer to FIG. 5, in this embodiment (the fourth embodiment), a sacrificial layer is deposited or coated on a surface of the substrate 10. After holes corresponding to the input and the output positions being formed on the sacrificial layer, deposit passivation materials on a surface of the substrate 10 firstly to form a first passivation layer 30. Remove the passivation material on the holes. Then perform the second time deposition of passivation materials to form a second passivation layer 60 after formation of the first electrode layer 40 and the second electrode layer 50. The second passivation layer 60 is disposed on and covering the first electrode layer 40 and the second electrode layer 50. Next carry out the third time deposition of passivation materials to form a third passivation layer 80 after deposition of the conductive layer 70. Then, remove a part of the passivation materials on the first electrode layer 40, the second electrode layer 50, and the conductive layer 70, and dispose the first connection electrode layer 92 and the second connection electrode layer 94 thereover. At final, remove the sacrificial layer to complete production of the micro heating and sensing device 1 in which the passivation layer 110 covers the first electrode layer 40, the second electrode layer 50, and the conductive layer 70.

A material for the passivation layer 110 is selected from the group consisting of aluminum oxide (Al₂O₃), silicon nitride (Si₃N₄), silicon dioxide (SiO₂), titanium dioxide (TiO₂), and a combination thereof.

In conventional micro heating and sensing device, an outer surface of the metal electrode is covered with insulation passivation layer made of insulation nitrides or oxides which are difficult to react with air and impurities outside and having lower thermal conductivity. These materials protect the electrodes and further address the electrode poisoning issue.

The same as the third embodiment, one end of the metal electrode or the whole metal electrode is replaced by conductive oxides or conductive nitrides in the fourth embodiment. Thereby the process to deposit passivation layers on the metal electrode on the surface of the micro heating and sensing device for protection can be omitted.

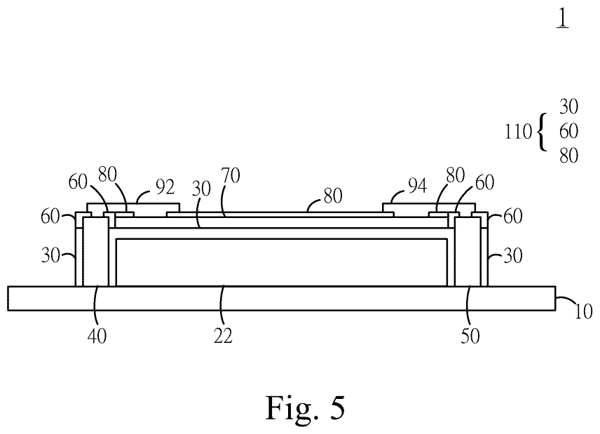

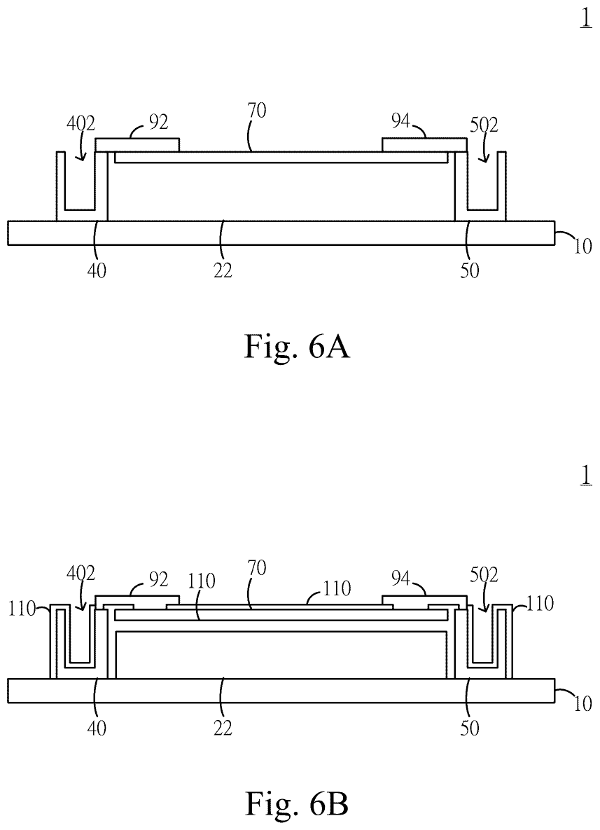

The metal electrode itself is a good thermal conductor. In order to reduce effects of heat loss of the metal electrode on the micro heating and sensing device, sectional views of a fifth embodiment formed based on the third and the fourth embodiments and shown in FIG. 6A and FIG. 6B are provided. In the fifth embodiment, a lower part of the first electrode layer 40 of the micro heating and sensing device 1 is extending horizontally toward a first side and then extending upward while a lower part of the second electrode layer 50 of the micro heating and sensing device 1 is extending horizontally toward a second side and then extending upward.

Refer to FIG. 6A and FIG. 6B, a fifth embodiment is provided. Besides one end of the metal electrode or the whole metal electrode being replaced by conductive oxides or conductive nitrides, the first electrode layer 40 and the second electrode layer 50 are further etched respectively to have a first recess 402 and a second recess 502 on the electrode layer for further reduction of heat loss caused by the thermal conductive electrode, like the air layer 22 mentioned above.

Compared with the third and the fourth embodiments in which one end of the metal electrode or the whole metal electrode is replaced by conductive oxides or conductive nitrides and covering with the passivation layers, the fifth embodiment is further provided with the recesses on the electrodes to reduce heat loss caused by the conductive electrode and further reduce effects of the heat loss on the micro heating and sensing device. Thus, the micro heating and sensing device's ability of temperature control and thermal retention is improved.

In summary, the present invention uses conductive oxides or conductive nitrides to replace metals on one end of the metal electrodes or the whole metal electrodes. Thereby not only the problem of the micro heating and sensing device available now that the passivation layer must be disposed on the rear end of the electrodes can be solved, process cost and production time are also both reduced. Poor contact between the electrode and the conductive layer can be reduced by using different electrode structures. The temperature control ability and thermal insulation effect are improved. The passivation layers are used to prevent interactions between the electrode and external gases or impurities.

The present invention meets requirements for patentability including novelty, non-obviousness and usefulness.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

1. A method of manufacturing a micro heating and sensing device comprising the following steps of:
disposing a sacrificial layer on a substrate;
etching the sacrificial layer to form a first hole and a second hole spaced apart from each other and both passing through the sacrificial layer toward an upper surface of the substrate;
disposing a first passivation layer on the sacrificial layer;
removing parts of the first passivation layer in part of the first hole and in part of the second hole by etching, and disposing a first electrode layer and a second electrode layer in the first hole and the second hole respectively to respectively extend outwardly excessing the first passivation layer on the sacrificial layer;
disposing and covering a second passivation layer on the first electrode layer and the second electrode layer;
disposing a conductive layer on an interval space between the first electrode layer and the second electrode layer and the conductive layer located on the first passivation layer;
disposing and covering a third passivation layer on the conductive layer;
removing part of the third passivation layer on the conductive layer by etching to form a first port and a second port;
removing part of the second passivation layer on the first electrode layer and the second electrode layer respectively by etching to form a third port and a fourth port;
separately disposing a first connection electrode layer and a second connection electrode layer on both the second passivation layer and the third passivation layer, the first connection electrode layer being coupled to the conductive layer and the first electrode layer respectively through the first port and the third port, the second connection electrode layer being coupled to the conductive layer and the second electrode layer respectively through the second port and the fourth port; and
removing the sacrificial layer, the first passivation layer located outside the first hole and covering the sacrificial layer, and the first passivation layer located outside the second hole and covering the sacrificial layer by etching;
wherein the conductive layer is configured for heating or sensing.

2. The method as claimed in claim 1, wherein the step of removing the first passivation layer on a bottom of the first hole and a bottom of the second hole by etching, and disposing a first electrode layer and a second electrode layer respectively on the first hole and the second hole further includes a step of:
etching the first electrode layer and the second electrode layer respectively to form a first recess and a second recess.

3. The method as claimed in claim 1, wherein a material for the first connection electrode layer and the second connection electrode layer is selected from the group consisting of indium tin oxide (ITO), zinc oxide (ZnO), indium zinc oxide (IZO), aluminum gallium oxide (AGO), aluminum-doped zinc oxide (AZO), titanium nitride (TiN), tantalum nitride (TaN), aluminum (Al), titanium (Ti), and a combination thereof.

4. The method as claimed in claim 1, wherein a material for both the first electrode layer and the second electrode layer is selected from the group consisting of gold (Ag), silver (Au), platinum (Pt), aluminum (Al), titanium (Ti), tantalum (Ta), indium tin oxide (ITO), zinc oxide (ZnO), indium zinc oxide (IZO), aluminum gallium oxide (AGO), aluminum-doped zinc oxide (AZO), titanium nitride (TiN), tantalum nitride (TaN), and a combination thereof.

5. The method as claimed in claim 1, wherein after the step of removing the sacrificial layer, the first passivation layer located outside the first hole and covering the sacrificial layer, and the first passivation layer located outside the second hole and covering the sacrificial layer by etching, an air layer is formed under the conductive layer and between the conductive layer and the substrate.

6. A micro heating and sensing device comprising:
a substrate;
a first electrode layer, disposed on an upper surface of the substrate;
a connection electrode layer, disposed at and coupled to an upper surface of the first electrode layer and extending toward one side horizontally;
a conductive layer, disposed on and coupled to a lower surface of the first connection electrode layer and extending toward the side horizontally; the conductive layer and the first electrode layer spaced apart from each other; an air layer formed between the conductive layer and the substrate;
a second connection electrode layer, disposed at and coupled to an upper surface of the conductive layer and extending toward the side horizontally; and
a second electrode layer, not only mounted to an upper surface of the substrate but also disposed on and coupled to a lower surface of the second connection electrode layer;
wherein a current is flowing from the first electrode layer to the first connection electrode layer, then through the conductive layer, the second connection electrode layer, and the second electrode layer in sequence, and lastly flowing to the substrate during electrical conduction process.

7. The device as claimed in claim 6, wherein the first connection electrode layer and the second connection electrode layer are made of a material selected from the group consisting of indium tin oxide (ITO), zinc oxide (ZnO), indium zinc oxide (IZO), aluminum gallium oxide (AGO), aluminum-doped zinc oxide (AZO), titanium nitride (TiN), tantalum nitride (TaN), aluminum (Al), titanium (Ti), and a combination thereof.

8. The device as claimed in claim 6, wherein both the first electrode layer and the second electrode layer are made of a material selected from the group consisting of gold (Ag), silver (Au), platinum (Pt), aluminum (Al), titanium (Ti), tantalum (Ta), indium tin oxide (ITO), zinc oxide (ZnO), indium zinc oxide (IZO), aluminum gallium oxide (AGO), aluminum-doped zinc oxide (AZO), titanium nitride (TiN), tantalum nitride (TaN), and a combination thereof.

9. The device as claimed in claim 6, wherein the micro heating and sensing device further includes a passivation layer which is made of insulation materials and covering the first electrode layer, the second electrode layer, and the conductive layer; the insulation material for the passivation layer is selected from the group consisting of aluminum oxide (Al₂O₃), silicon nitride (Si₃N₄), silicon dioxide (SiO₂), titanium dioxide (TiO₂), and a combination thereof.

10. The device as claimed in claim 6, wherein a lower part of the first electrode layer is extending horizontally toward a first side and then extending upward (to form a first recess) while a lower part of the second electrode layer is extending horizontally toward a second side and then extending upward (to form a second recess).
